(19) United States
(12) Patent Application Publication
(10) Pub. No.: US 2019/0062260 A1
(43) Pub. Date: Feb. 28, 2019

(54) ANTIMICROBIAL COMPOSITIONS, METHODS OF MAKING, AND METHODS OF USE

(71) Applicants: Rohm and Haas Company, Collegeville, PA (US); Dow Global Technologies LLC, Midland, MI (US); The Board of Trustees of the University of Illinois, Urbana, IL (US)

(72) Inventors: Keith Harris, Midland, MI (US); Joshua S. Katz, Merion Station, PA (US); David M. Laganella, Swedesboro, NJ (US); Shampa R. Samanta, Urbana, IL (US); Brittany A. Walker, Urbana, IL (US); Steven C. Zimmerman, Champaign, IL (US)

(21) Appl. No.: 16/117,418

(22) Filed: Aug. 30, 2018

Related U.S. Application Data
(60) Provisional application No. 62/552,676, filed on Aug. 31, 2017.

(51) Int. Cl. C07C 69/73; C07C 67/287; C08L 71/02

(52) U.S. Cl. CPC C07C 69/73; C08L 71/02; C07C 67/287

(57) ABSTRACT

The invention provides a protected antimicrobial compound and methods of using the same.

The invention provides a protected antimicrobial compound and methods of using the same.

For oilfield chemical systems, reactive components are often part of the design, and most of time their reactivity needs to be delayed or suppressed until the right time for optimal efficacy. This may be due to a variety of reasons including but not limited to different performance requirements at different stages of oilfield operations and also for toxicological and environmental hazard reasons. Acrolein, one such example of a reactive compound, is a potent biocide and sulfide scavenger but is also a highly-toxic chemical. Consequently, it is not widely employed as an industrial biocide in order to not expose users to its toxicity. The present invention solves the problem of the art by providing less-toxic materials that can release acrolein at the point of use. Compared to acrolein, the present invention will ease costs of shipping and reduce health and safety risks while maintaining antimicrobial performance.

The present invention provides a protected antimicrobial composition comprising:

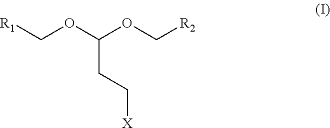

wherein X is Cl, Br, I, tosyl, F, or a conjugate base of a strong acid; and, R₁ and R₂ are aliphatic or aromatic organic substituent groups independently having from 1-20 carbons or a polymerizable functional group selected from the group consisting of (meth)acrylate esters, (meth)acrylamides, and vinyl containing groups.

The present invention further provides a method of using the protected antimicrobial composition by contacting it with an environment having a pH of 7 or less to release acrolein. A method of using the protected antimicrobial composition by adding it to an oil well; water reservoir; natural gas reservoir; paint; film; pressure treated wood; coating; caulk; wall board; or plastic is also provided.

As used herein, the term “strong acid” means an acid that is fully dissociated in solution, typically with a pH≤1 at a concentration of 1M.

The protected antimicrobial composition of the present invention is represented by the following structure:

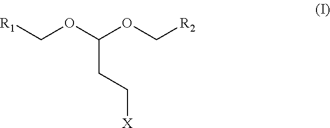

wherein X is Cl, Br, I, tosyl, F, or a conjugate base of a strong acid; and,
R₁ and R₂ are aliphatic or aromatic organic substituent groups independently having from 1-20 carbons, optionally additionally containing oxygen, nitrogen, sulfur, or combinations thereof, and wherein any metal present is present in a trace amount. Examples of suitable functional groups for R₁ and R₂ are ether, polyether, alkene and alkyne groups. Alternatively, R₁ and/or R₂ include a polymerizable functional group selected from (meth)acrylate esters; (meth)acrylamides; vinyl containing groups such as vinyl ethers or styrene moieties. R₁ and R₂ may be the same functional group or may be different. Suitably, X is I, Br, or Cl. More Suitably X is I or Br.

More specifically, the protected antimicrobial composition of the present invention may be represented by the following structure:

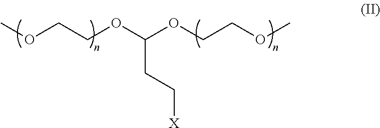

wherein X is Cl, Br, I, tosyl, F, or a conjugate base of a strong acid and further wherein n is between 1 and 100, alternatively between 2 and 5, and further alternatively n is 3.

The protected antimicrobial composition of the present invention may further have the following structure:

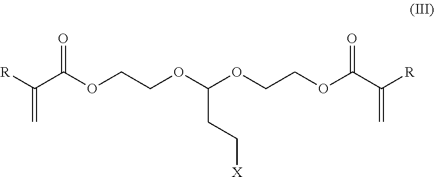

wherein X is Cl, Br, I, tosyl, F, or a conjugate base of a strong acid and further wherein R is CH₃ or H.

When any of the protected antimicrobial compositions of structures I, II, or III are contacted with an environment having a pH of 7 or less, alternatively 6 or less, alternatively 5 or less, alternatively 4 or less, alternatively 3 or less, or alternatively 2 or less, acrolein is released. The environment may be at the aforementioned pH prior to the addition of the protected antimicrobial composition or may be adjusted to the aforementioned pH post addition of the protected antimicrobial composition. Environments of the present invention may be any gaseous, liquid, or solid medium, or mixtures thereof. Particularly suitable environments for the present invention are aqueous environments. Environments of the present invention may have temperatures ranging from −20 to 200, alternatively 0-120, and further alternatively 15 to 80 degrees Celsius.

Protected antimicrobial compositions of the present invention may be used in a variety of applications including but not limited to an oil well; water reservoir; natural gas reservoir; paint; film; pressure treated wood; coating; caulk; wall board; or plastic. Typically, the protected antimicrobial compositions would be added directly as a component of any of the aforementioned applications, alternatively the protected antimicrobial composition may be formulated with other materials, including additional antimicrobial agents, to create protected antimicrobial formulations.

### EXAMPLES

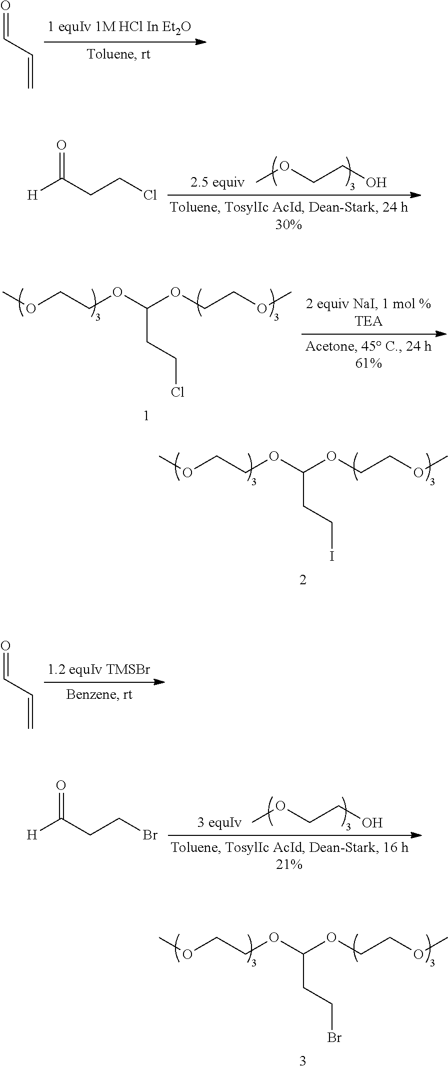

### Synthesis of Molecules

Materials: Unless otherwise noted, all the solvents were ACS reagent grade (conforms to specifications defined by the Committee on Analytical Reagents of the American Chemical Society) and purchased from Acros Organics, Fisher Scientific, Macron Fine Chemicals, or Sigma-Aldrich, and used without further purification. Acrolein was purchased from Thermo Fischer Scientific Chemicals INC/Alfa Aesar and used as received. Triethylene glycol monomethyl ether, TsOH.H₂O, NaI were purchased from Aldrich and used as received. Bromotrimethylsilane (Oakwood Chemicals), and 1 M HCl in diethylether (Alfa Aesar) were used as received.

Compound 1: Into a 500 mL round bottom flask (RBF) was added 200 mL toluene, 50 mL diethyl ether, and 6.64 mL (0.1 mol) acrolein. The RBF was fitted with a liquid addition funnel under N₂ atmosphere and 100 mL of 1 M HCl in diethyl ether was added dropwise over 1 h at room temperature (˜25° C.) while stirring. The crude reaction mixture indicated ˜99% conversion of the acrolein to 3-chloropropanaldehyde by ¹H NMR (500 MHz, CDCl₃). Atmospheric distillation was used to remove ˜150 mL of diethyl ether from the reaction crude. To the RBF was added 40 mL (0.25 mol) triethylene glycol monomethyl ether and 230 mg (1.2 mmol) TsOH.H₂O. The solution was refluxed for 24 h under Dean-stark conditions. At the end of the reaction, solvent was removed under reduced pressure, the crude was washed with 100 mL of hexane, and re-dissolved in 100 mL of ethyl acetate. This solution was washed with 300 mL of an aqueous solution containing 5 wt. % K₂CO₃ and 10 wt. % NaCl. The product was back extracted with 2×100 mL of ethyl acetate. The organic layers were combined, washed with 150 mL brine, and dried over Na₂SO₄. The solution was vacuum filtered and concentrated under reduced pressure to obtain 26 g of 1 as crude product, 7 g of which was used directly in the Finkelstein reaction, and the remaining crude was purified by silica column chromatography using 30% acetone in petroleum ether (v/v) with <1% triethylamine to isolate yellow liquid product. Yield: 12.19 g, 30%.

Compound 2: 1 was converted to 2 via Finkelstein reaction. In a 100 ml round bottom flask with condenser, the precursor (7 g, 17.4 mmol) was dissolved in 60 mL of acetone and treated with 5.68 g, ˜2 equivalents, of NaI and 1 mol % triethylamine at 45° C. for 24 h. At the end of the reaction, the crude was transferred to a 250 mL Erlenmeyer flask and 60 mL of diethyl ether was added to this solution while cooling the solution at −20° C. to precipitate out most of the remaining NaI and NaCl. This cold solution was quickly filtered off through a sintered crucible. The filtrate was concentrated under reduced pressure, re-dissolved in 60 mL of ethyl acetate, and washed with 2×30 mL of 10% Na₂S₂O₃ solution. The aqueous solution was back extracted with 30 mL ethyl acetate. All organic layers were combined, washed with 100 mL of brine, and dried over Na₂SO₄. The solution was vacuum filtered and concentrated under reduced pressure. The product was further purified by silica column chromatography using 30% acetone in petroleum ether (v/v) with <1% triethylamine to isolate yellow liquid product. Yield: 5.24 g, 61%.

Compound 3: Into a 250 mL round bottom flask (RBF) cooled to 0° C. was added 125 mL dry benzene and 6.64 mL (0.1 mol) acrolein. The RBF was fitted with a septum and 15.8 mL (0.12 mol) trimethylsilyl bromide was added dropwise over 5 min. The solution stirred for 30 min at 0° C., warmed to room temperature, and stirred for an additional 2 h prior to the addition of 48 mL (0.30 mol) triethylene glycol monomethyl ether and 500 mg (2.6 mmol) TsOH.H₂O. The mixture was refluxed under a Dean-Stark trap for 16 h, cooled, and evaporated. Chromatography of the residue on silica gel (elution with 3:1 petroleum ether/acetone) gave 3 as a yellow liquid. Yield: 9.5 g, 21%.

### Synthesis of Compound III

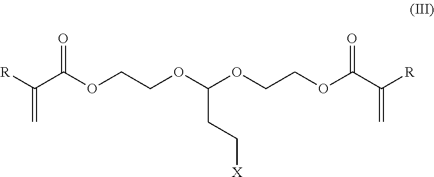

where X is I and R is H.

In a 2 L round bottom flask with 100 g activated 3 Å molecular sieves, 900 mL HPLC-grade MeCN was added under N₂. NaI (128 g, 0.85 mol) was added under N₂, followed by addition of acrolein (52 mL, 0.78 mol). The solution was added to trimethylsilyl chloride (110 mL, 0.87 mnol) over 15 min. via an additional funnel. The solution was stirred at room temperature for 2 h, followed by addition of 2-hydroxyethyl acrylate (204.4 mL, 1.78 mol). The reaction was stirred for another 2 h.

The solution was filtered through sintered crucible, ethyl acetate was used to rinse the collected solids. The filtrate was rotovapped to dryness, pumped on high vacuum, and re-dissolved in 60% ethyl acetate/hexanes (500 mL). The solution was cooled to −20 ° C. for 2 h, filtered off solids in the suspension, and washed with saturated NaHCO₃ (250 mL×2), 10% w/v Na₂S₂O₃ (250 mL×2), DI H₂O (250 mL×3). The layers are separated and the aqueous layer was back extracted with 30% ethyl acetate/hexanes (250 mL×1). The organic layers were combined, washed with brine (250 mL×1), dried over Na₂SO₄ for 2 h, filtered to remove the drying agent, and evaporated on rotovap. The obtained oil was applied on top of a 20% ethyl acetate/hexanes-wetted plug consist of 50 mL celite at bottom and 50 mL basic alumina at top in a 200 mL sintered crucible. Vacuum was applied to elute the product with 1.5 L of 20% ethyl acetate/hexanes, until no intense product spot observed on thin-layer chromatography (TLC). The eluted solution was rotovapped and pumped on high vacuum to remove excess solvent.

### Synthesis of an Acrylic Polymer Containing (III)

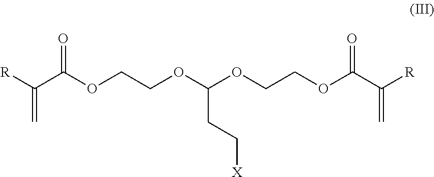

where X is I and R is H.

3 g of a solution of 48% styrene, 40% butyl acrylate, 10% the compound above, and 2% acrylic acid were added to 7 g of a 1% sodium dodecyl sulfate in 0.1M phosphate buffer solution. The two phases were vortexed to form an emulsion. 200 mg of (NH₄)₂S₂O₈ and 270 mg of (NH₄)₂S₂O₅ were added to initiate polymerization. The reaction was allowed to proceed under nitrogen for 2 hrs. at room temperature followed by 2 hrs. at 70° C., yielding the polymer.

### Degradation of Compound and Release of Acrolein

For compound

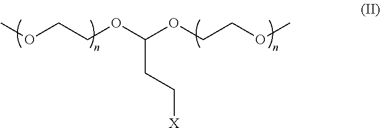

n=3, X as defined in the table. Measured at 25 degrees Celsius (room temperature), tracked via NMR spectroscopy. All compounds were prepared at 50 mM in deuterated water, pH adjusted with deuterium chloride.

| | | % | % acrolein | % acrolein |
|---|---|---|---|---|
| | % degraded pH | degraded pH | generated pH 7, | generated pH 3, |
| X | 7, 7 days | 3, 3 days | 7 days | 3 days |
| Cl | 0 | 50 | 0 | 1 |
| Br | 95 | 100 | 12 | 8 |
| I | 40 | 80 | 6 | 7 |

For compound

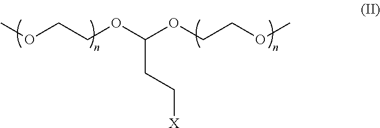

n=3, X is Br. Measured at 25 or 70 degrees Celsius, tracked via NMR spectroscopy. All compounds were prepared at 50 mM in deuterated water at pH 7. Measurements were made after 4 hours.

| % degraded | % degraded | % acrolein | % acrolein |
|---|---|---|---|
| 25° C. | 70° C. | generated 25° C. | generated 70° C. |
| 0 | 85 | 0 | 40 |

We claim:

1. A protected antimicrobial composition comprising: 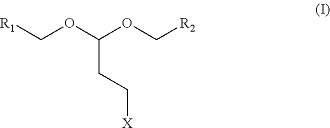
wherein X is Cl, Br, I, tosyl, F, or a conjugate base of a strong acid; and, R₁ and R₂ are aliphatic or aromatic organic substituent groups independently having from 1-20 carbons or a polymerizable functional group selected from the group consisting of (meth)acrylate esters, (meth)acrylamides, and vinyl containing groups.

2. The protected antimicrobial composition of claim 1 comprising the following structure: 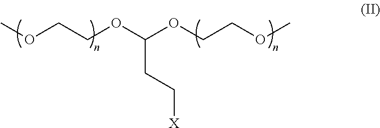
further wherein n is between 1 and 100.

3. The protected antimicrobial composition of claim 2 wherein n is between 2 and 5.

4. The protected antimicrobial composition of claim 2 wherein X is Br.

5. The protected antimicrobial composition of claim 2 wherein X is I.

6. The protected antimicrobial composition of claim 1 comprising the following structure: 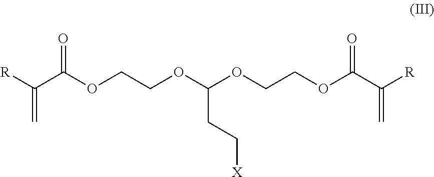
wherein R is CH₃ or H.

7. A formulation comprising the protected antimicrobial composition of claim 1.

8. A method of using the antimicrobial composition of claim 1 comprising:
i. providing the protected antimicrobial composition of claim 1; and
ii. contacting the protected antimicrobial composition with an environment having a pH of 7 or less to release acrolein.

9. The method of claim 8 wherein the temperature of the environment is from 0 to 200 C.

10. A method of using the compound of claim 1 wherein the protected antimicrobial composition of claim 1 is added to an oil well; water reservoir; natural gas reservoir; paint; film; pressure treated wood; coating; caulk; wall board; or plastic.
